(19) United States
(12) Patent Application Publication
(10) Pub. No.: US 2026/0157983 A1
(43) Pub. Date: Jun. 11, 2026

(54) DOFETILIDE FORMULATIONS

(71) Applicant: Hyloris Developments SA, Liège (BE)

(72) Inventor: Atul Patil, Liège (BE)

(21) Appl. No.: 19/356,446

(22) Filed: Oct. 13, 2025

Related U.S. Application Data
(60) Provisional application No. 63/706,764, filed on Oct. 14, 2024.

(51) Int. Cl. A61K 31/145; A61K 9/00; A61K 47/02; A61K 47/12

(52) U.S. Cl. CPC A61K 31/145; A61K 9/0019; A61K 47/02; A61K 47/12

(57) ABSTRACT

The present invention relates to novel dofetilide-containing pharmaceutical formulations, particularly those that contain sodium chloride, sodium acetate, and acetic acid. The present invention further relates to dilutions of these formulations in normal saline or dextrose solutions. The present invention also provides a method of manufacturing pharmaceutical formulations and a method of treatment with the pharmaceutical formulations and dilutions thereof.

### CROSS REFERENCE TO RELATED APPLICATIONS

This application claims priority to U.S. Provisional Application No. 63/706,764, filed 14 Oct. 2024, the disclosure of which is hereby incorporated by reference in its entirety.

### FIELD OF THE INVENTION

The present invention relates to novel formulations of dofetilide and their manufacturing and use.

### BACKGROUND OF THE INVENTION

Dofetilide (β-((p-methanesulfonamidophenethyl)methylamino) methanesulfono-p-phenetidide) is an anti-arrhythmic of the Vaughn Williams Class III. dofetilide is commercially available as an oral capsule, with Tykosin® by Pfizer for the maintenance of sinus rhythm in individuals prone to the formation of atrial fibrillation and flutter, and for the chemical cardioversion to sinus rhythm from atrial fibrillation and flutter. However, patients under nothing by mouth (NPO), or having emesis, severe gastrointestinal (GI) absorption problems, are intubated, or who have problems swallowing, cannot take dofetilide orally. To avoid these complications, and to enhance the patient compliance it would be advantageous to develop a dofetilide parenteral dosage form. Currently there is no commercially approved sterile solution for infusion for dofetilide.

As discussed in U.S. Pat. No. 11,364,213, dofetilide is currently being investigated for use intravenously. However, U.S. Pat. No. 11,364,213 compositions either use excipients that have an unfavorable toxicity profile or have an acidic pH (e.g., lower than 5).

Atrial fibrillation, long considered the most common condition leading to an irregular heartbeat, is a growing and serious global health problem. Hence, there is a need for additional treatment options.

Thus, it would be beneficial to discover formulations of dofetilide that would allow for intravenous administration For intravenous administration it can be beneficial to have storage stable, ready-to-dilute or ready-to-use dofetilide formulations. In addition, it could be advantageous to have dofetilide formulations ready-to-dilute or ready-to-use for one-day loading and methods of one-day dofetilide loading.

### SUMMARY OF THE INVENTION

Accordingly, in an aspect, the present invention describes novel pharmaceutical compositions, comprising: a therapeutically effective amount of dofetilide and a pharmaceutically acceptable carrier.

In another aspect, the formulations described herein are suitable for intravenous administration.

These and other objects, which will become apparent during the following detailed description, have been achieved by the inventors' discovery of dofetilide formulations.

### DETAILED DESCRIPTION OF THE INVENTION

All references cited herein are hereby incorporated in their entirety by reference.

The inventors have surprisingly found that a composition according to an embodiment of the invention is advantageous as it provides a dofetilide composition with long-term stability and provided for intravenous administration. The latter means that the pH and preferably also osmolality values are close to the physiological blood/plasma levels (e.g., typically 275-295 mOsm/kg).

Provided for intravenous administration means that the composition needs to fulfill some specific physiological conditions to ensure safety, compatibility, and efficacy when administered intravenously such as having pH and osmolality values similar to the physiological blood/plasma levels. In addition, the composition is sterile.

Thus, in an aspect, the present invention relates to novel dofetilide formulations.

In another aspect, the present invention relates to a novel pharmaceutical composition, comprising:
a. 10-1000 μg/mL dofetilide;
b. 4-8 mg/mL sodium chloride;
c. 0.1-0.5 mg/mL acetic acid;
d. 2-6 mg/mL sodium acetate; and
e. water;
wherein:
   the pH of the composition is from 4-7; and,
   the composition is suitable for intravenous administration.

From refers to values that are inclusive of the endpoints (e.g., from 4-7 include both 4 and 7 in the recited range).

Additional examples of the concentration of dofetilide include: (a) 10, 20, 30, 40, 50, 60, 70, 80 90, 100, 110, 120, 130, 140, 150, 160, 170, 180, 190, 200, 220, 240, 260, 280, 300, 320, 340, 360, 380, 400, 420, 440, 460, 480, and 500 μg/mL, (b) 10-500 μg/mL, (c) 10-100 μg/mL, (d) 20-80 μg/mL, (e) 30-70 g/mL, (f) 40-60 μg/mL, and (g) 50 μg/mL.

Additional examples of the concentration of sodium chloride include: (a) 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, and 8 mg/mL; (b) 5-8 mg/mL; (c) 6-7 mg/mL, (d) 6.3-6.7 mg/mL; and (e) 6.5 mg/mL.

Additional examples of the concentration of acetic acid include: (a) 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, and 0.5 mg/mL; (b) 0.2-0.4 mg/mL; (c) 0.25-0.35 mg/mL; (d) 0.3-0.35 mg/mL; and, (e) 0.31 mg/mL.

Additional examples of the concentration of sodium acetate include: (a) 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, and 6 mg/mL; (b) 3-5 mg/mL; (c) 3.5-4.5 mg/mL; (d) 3.5-4 mg/mL; and, (e) 3.7 mg/mL.

Examples of the pH of the intravenous formulation include (a) 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, and 7; (b) 5-6; (c) 5.2-5.8, (d) 5.4-5.6; and, (e) 5.5.

In an aspect, the present invention relates to a novel composition that complies with IIG limits, which facilitates regulatory approval. IIG=Inactive Ingredient Database (FDA). The IIG specifies the maximum amount of inactive ingredient for a route of administration/dosage form.

In another aspect, the present invention relates to a novel composition, wherein from 1.1×10⁻⁹ to 0.0003 M HCl is present. Additional examples of the amount of HCl present include 1.1×10⁻⁹ to 0.00029M, 1.1×10⁻⁹ to 0.00028M, 1.1×10⁻⁹ to 0.00027M, 1.1×10⁻⁹ to 0.00026M, and 1.1×10⁻⁹ to 0.00025M.

In another aspect, the present invention relates to a novel pharmaceutical composition, comprising:
a. 10-500 μg/mL dofetilide;
b. 5-8 mg/mL sodium chloride;
c. 0.2-0.4 mg/mL acetic acid;
d. 3-5 mg/mL sodium acetate; and
e. water;
wherein:
   the pH of the composition is from 5-6; and,
   the composition is suitable for intravenous administration.

In another aspect, the present invention relates to a novel pharmaceutical composition, comprising:
a. 10-100 μg/mL dofetilide;
b. 6-7 mg/mL sodium chloride;
c. 0.25-0.35 mg/mL acetic acid;
d. 3.5-4.5 mg/mL sodium acetate; and
e. water;
wherein:
   the pH of the composition is from 5.2-5.8; and,
   the composition is suitable for intravenous administration.

In another aspect, the present invention relates to a novel pharmaceutical composition, comprising:
a. 40-60 μg/mL dofetilide;
b. 6.3-6.7 mg/mL sodium chloride;
c. 0.3-0.35 mg/mL acetic acid;
d. 3.5-4 mg/mL sodium acetate; and
e. water;
wherein:
   the pH of the composition is from 5.4-5.6; and,
   the composition is suitable for intravenous administration.

In another aspect, the present invention relates to a novel pharmaceutical composition, comprising:
a. 50 μg/mL dofetilide;
b. 6.5 mg/mL sodium chloride;
c. 0.31 mg/mL acetic acid;
d. 3.7 mg/mL sodium acetate; and
e. water;
wherein:
   the pH of the composition is 5.5; and,
   the composition is suitable for intravenous administration.

In another aspect, the present invention relates to a novel pharmaceutical composition, consisting essentially of:
a) 50 μg/mL dofetilide;
b) 6.5 mg/mL sodium chloride;
c) 0.31 mg/mL acetic acid;
d) 3.7 mg/mL sodium acetate; and,
e) water;
wherein:
   less than 1.00% w/w dofetilide related compound A (USP) is present;
   the pH of the composition is from 5.4-5.6 and is optionally obtained by the addition of NaOH or HCl; and,
   the composition is suitable for intravenous administration.

In another aspect, the present invention relates to a novel pharmaceutical composition, consists of:
a) 50 μg/mL dofetilide;
b) 6.5 mg/mL sodium chloride;
c) 0.31 mg/mL acetic acid;
d) 3.7 mg/mL sodium acetate; and,
e) water;
wherein:
   less than 1.00% w/w dofetilide related compound A (USP) is present;
   the pH of the composition is from 5.4-5.6 and is optionally obtained by the addition of NaOH or HCl; and,
   the composition is suitable for intravenous administration.

In another aspect, the present invention relates to compositions which are substantially free of surfactants (e.g., to solubilize dofetilide). By “free of surfactant” as used herein is meant, below a detection limit of 0.03 microgram/mL.

In another aspect, the present invention relates to compositions which are substantially free of oils (e.g., to emulsify dofetilide). By “free of oil” as used herein is meant, below a detection limit of 0.03 microgram/mL.

In another aspect, the present invention relates to compositions that are solutions.

In another aspect, the present invention relates to compositions that are substantially free of suspended materials. This means solution clarity.

In another aspect, the present invention relates to compositions that are suitable for IV administration.

In another aspect, the present invention relates to compositions having an osmolality of from 285-320 mOsmol/L as determined by point depression according to USP 788. Additional examples of osmolality (mOsmol/L) include (a) 285, 290, 295, 300, 305, 310, 315, and 320, (b) 290-315, (c) 295-310, and (d) 300-305.

In another aspect, the present invention relates to compositions having a storage stability of at least 6 months as measured at 40° C. and 75% Relative Humidity.

In another aspect, the present invention relates to compositions having a dofetilide related compound A below 1% w/w. dofetilide related compound A is known as (N-[4-(2-{2-[4-Methanesulfonamido) phenoxy]ethylamino}ethyl)phenyl]methanesulfonamide (CAS number 176447-94-2; United States Pharmacopeia). Additional examples include less than 0.9, 0.7, and 0.5% w/w.

In another aspect, the present invention relates to compositions having a storage stability of at least 6 months, at least 12 months, at least 18 months, and at least 24 months, as measured at 40° C. and 75% Relative Humidity.

In another aspect, the present invention relates to compositions having a storage stability of at least 6 months, at least 12 months, at least 18 months, and at least 24 months, as measured at 25° C. and 60% Relative Humidity.

In another aspect, the present invention relates to a method of manufacturing compositions according to the invention, comprising the step of using dofetilide active ingredient with particle size D(90) of 10 microns. Additional examples include particles sizes less than 10, 9.5, 9, 8.5, 8, 7.5, 7, 6.5, 6, 5.5, 5, 4.5, 4, 3.5, 3, 2.5, and 2 microns.

In another aspect, the present invention relates to a method of manufacturing dofetilide compositions according to the invention, comprising dofetilide in polymorph P162 form. The polymorph P162 form is discussed in EP1027329 and U.S. Pat. No. 36,124,363.

In another aspect, the present invention relates to a method of manufacturing dofetilide composition according to the invention, comprising aseptic filtration of the composition.

In another aspect, the present invention relates to a composition that is aseptically filtrated.

In another aspect, the present invention relates to a method of treatment of a patient in need of dofetilide therapy, comprising the step of providing a composition described herein; diluting the composition in normal saline 0.09% or dextrose 5% prior to intravenous administration to said patient.

In another aspect, the diluted composition has an osmolality of 285-320 mOsmol/L as determined by point depression according to USP 788. Additional examples of osmolality include (a) 285, 290, 295, 300, 305, 310, 315, and 320, (b) 290-315, (c) 295-310, and (d) 300-305.

In another aspect, the present invention relates to a method of treating a patient in need of dofetilide therapy, comprising: administering a composition described herein to maintain the normal sinus rhythm in a patient prone to the formation of atrial fibrillation and/or flutter. In another aspect, the administration is intravenous administration.

In another aspect, the present invention relates to a method of treating a patient in need of dofetilide therapy, comprising: administering a composition described herein for chemical cardioversion to normal sinus rhythm from atrial fibrillation and/or flutter. In another aspect, the administration is intravenous administration.

In another aspect, the patient is a human patient.

In another aspect, the human patient was not previously on dofetilide oral tablets.

In another aspect, the intravenous administration is preceded by diluting in saline 0.09% or dextrose 5% a composition according to an aspect of the invention.

In another aspect, the present invention relates to a diluted dofetilide composition for use in a patient in need of dofetilide therapy formed by diluting a composition described herein in saline 0.09% or dextrose 5% prior to intravenous administration of the diluted dofetilide composition to the patient.

In another aspect, the present invention relates to a container, comprising: a composition described herein, wherein the container is an amber colored glass container. Amber colored glass protects the composition from UV rays below 450 nm.

In another aspect, the present invention relates to a container, comprising: a composition described herein, wherein the container is an infusion bag. In another aspect, the infusion bag is an amber bag. The amber bag is beneficial for restricting exposure of the composition to UV light.

In another aspect, the present invention the container described herein holds a unit dose of 5 to 10 mL of a sterile composition described herein, with a dofetilide concentration of 50 microgram per mL. This is advantageous as it provides a product that is ready-to-dilute or ready-to-use. This is especially beneficial for one-day loading and methods of one-day dofetilide loading.

In another aspect, the present invention relates to a method of manufacturing a composition described herein, comprising the step of providing dofetilide with a particle side D(90) of less than 10 microns. In another aspect, the dofetilide is polymorph P162 dofetilide. In another aspect, the composition is aseptically filtrated. In another aspect, the containers for receipt of the sterilized composition are pre-sterilized.

In another aspect, a composition described herein is sterile and comprises less than 25 EU/mL of bacterial endotoxin as determined by USP<85>.

The present invention may be embodied in other specific forms without departing from the spirit or essential attributes thereof. This invention encompasses all combinations of aspects of the invention noted herein. It is understood that any and all embodiments of the present invention may be taken in conjunction with any other embodiment or embodiments to describe additional embodiments. It is also to be understood that each individual element of the embodiments is intended to be taken individually as its own independent embodiment. Furthermore, any element of an embodiment is meant to be combined with any and all other elements from any embodiment to describe an additional embodiment.

Other features of the invention will become apparent in the course of the following descriptions of exemplary embodiments that are given for illustration of the invention and are not intended to be limiting thereof.

### EXAMPLES

Examples 1 and 2 as exemplified in the table below are manufactured at 2 different locations using different manufacturing processes. In example 1 the API is added first in the water and then rest of the excipients/buffers were mixed, while in Example 2 the API is added after the addition of buffers.

### Molecular Weight for Reference:

a. Acetic acid: 60.05
b. Hydrochloric acid: 36.5
c. Sodium acetate anhydrous: 82.03
d. Sodium Chloride: 58.44

### Examples 1 and 2

| | Formulation Example 1 | | Formulation Example 2 | |
|---|---|---|---|---|
| | Dofetilide 50 μg/ml | | Dofetilide 50 μg/ml | |
| | solution for injection | | solution for injection | |
| | 0.05M acetate buffer | | 0.05M acetate buffer | |
| | pH 5.5 | | pH 5.5 | |
| | | Batch Qty (mg) | | Batch Qty (mg) |
| | | Batch Size: | | Batch Size: |
| Ingredients | mg/mL | 400 mL | mg/mL | 400 mL |
| Dofetilide | 10.05 | 20.00 | 0.05 | 20.00 |
| (micronized) | | | | |
| Sodium Chloride | 6.50 | 2600.00 | 6.50 | 2600.00 |
| Glacial Acetic | 0.31 | 124.00 | 0.31 | 124.00 |
| Acid | | | | |
| Sodium Acetate, | 3.70 | 1480.00 | 3.70 | 1480.00 |
| Anhydrous | | | | |
| Water for | q.s* to 1 mL | q.s to 400 mL | q.s to 1 mL | q.s to 400 mL |
| Injection (WFI) | | | | |
| 50% NaOH | q.s | q.s to adjust pH | q.s | q.s to adjust pH |
| | | 5.4 to 5.6 | | 5.4 to 5.6 |
| 1N HCl | q.s | q.s to adjust pH | q.s | q.s to adjust pH |
| | | 5.4 to 5.6 | | 5.4 to 5.6 |

*q.s = quantum santis, as much as needed

### Example 1: Dofetilide 50 μg/Ml Solution for Injection in 0.05M Acetate Buffer pH 5.5

A 400 mL batch size was made as follows:
a. Step-1: In a suitable stainless steel/glass container a mixing speed is set to create a slight vortex in WFI with overhead mixer or suitable mixing apparatus.
b. Step-2: Take a sample for dissolved oxygen determination.
c. Step-3: While continuing to mix, add 20 mg of dofetilide to the stirring water and allow mixing for a minimum of 60 minutes. Record the amount added and record the addition and mixing time.
d. Step-4: While continuing to mix, add 2.60 g of Sodium Chloride to the stirring water and allow mixing for a minimum of 5 minutes.
e. Step-5: While continuing to mix, add 1.48 g of Sodium Acetate anhydrous to the stirring water and allow mixing for a minimum of 5 minutes.
f. Step-6: While continuing to mix, add 124 mg (0.188 mL) of Glacial Acetic Acid to the stirring water and allow mixing for a minimum 5 minutes.
g. Step-7: Weigh bulk container after addition of all excipients, transfer remaining water (to a total of 400 mL of solution) to bulk container.
h. Step-8: Mix for a minimum of 10 minutes.
i. Step-9: Measure the pH of the solution and record the value.
j. Step-10: Verify that the pH value is from 5.4 and 5.6.
k. Step-11: If the pH is less than 5.4, use 50% sodium hydroxide solution to adjust the pH, using a pipette and pipette tip to adjust the pH from 5.4 and 5.6.
l. Step-12: If the pH is greater than 5.6, take 1N hydrochloric acid using a sterile 5-mL syringe with needle to adjust the pH from 5.4 and 5.6.
m. Step-13: Filter the solution using a suitable syringe filter (0.22 μm). Take a sample for analysis of the specific gravity.
n. Step-14: Filter the solution again using a syringe filter prior to filling, fill the vials, and stopper them. Crimp the vials.

### Example 2: Dofetilide 50 μg/ml Solution for Injection in 0.05M Acetate Buffer pH 5.5

A 400 mL batch size was made as follows:
a. Step-1: In a suitable stainless-steel glass container a mixing speed is set so as to create a slight vortex in WFI with overhead mixer or suitable mixing apparatus.
b. Step-2: Take a sample for dissolved oxygen.
c. Step-3: While continuing to mix, add 2.60 g of Sodium Chloride to the stirring water and allow mixing for a minimum of 5 minutes.
d. Step-4: While continuing to mix, add 1.48 g of Sodium Acetate to the stirring water and allow mixing for a minimum of 5 minutes.
e. Step-5: While continuing to mix, add 124 (0.188 mL) mg of Glacial Acetic Acid to the stirring water and allow mixing for a minimum 5 minutes. Check the pH.
f. Step-6: While continuing to mix, add 20 mg of dofetilide to the stirring water and allow mixing for 60 minutes or till clear solution is obtained. Record the amount added and record addition and mixing time.
g. Step-7: Weigh bulk container after addition of all excipients, transfer remaining water (400 mL total solution) to bulk container.
h. Step-8: Mix for a minimum of 10 minutes.
i. Step-9: Measure the pH of the solution and record the value.
j. Step-10: Verify that the pH value is from 5.4 and 5.6.
k. Step-11: If the pH is less than 5.4, use 50% sodium hydroxide solution to adjust the pH, using a pipette and pipette tip to adjust the pH from 5.4 and 5.6.
l. Step-12: If the pH is greater than 5.6, take 1N hydrochloric acid using a sterile 5-mL. syringe with needle to adjust the pH from 5.4 and 5.6.
m. Step-13: Filter the solution using suitable syringe filter (0.22 μm). Take sample for specific gravity.
n. Step-14: Filter the solution again using syringe filter prior to filling, fill the vials, and stopper them. Crimp the vials.

### Example 3

### Primary Packing Materials for Both Formulation

| | |
|---|---|
| Glass vials | USP Type I, amber colored vials, 10R, |
| | USP Type I, clear vials, 10R |
| Rubber | Bromobutyl coated rubber stoppers |
| Stoppers | |

### Stability Design

| | 40 ± 2° C./ | | | | 25 ± 2° C./ | | | Control |
|---|---|---|---|---|---|---|---|---|
| | 75 ± 5% RH | | | | 60 ± 5% RH | | | Sample |
| Formulation | Time Points | | | | Time Points | | | 2-8° C./RT |
| Example -1 | 2 W^{@} | 4 W^{@} | 12 W^{@, $} | 24 W^{@, $} | 4 W^{@} | 12 W^{@, $} | 24 W^{@, $} | On |
| | | | | | | | | Demand |
| Example-2 | 2 W^{@} | 4 W^{@} | 12 W^{@, $} | 24 W^{@, $} | 4 W^{@} | 12 W^{@, $} | 24 W^{@, $} | On |
| | | | | | | | | Demand |

Orientation of sample: ^{@}Inverted, ^{$}Upright, W = Weeks.
Placebo to be kept for stability.
Note:
Samples only at accelerated stability conditions were analysed

### Stability Results

### Formulation Example 1

In this formulation, Example 1, the samples were withdrawn at different time points as per stability protocol and tested at 2 weeks to 24 weeks as indicated in first column. The second column indicates the accelerated stability condition of 40° C./75% RH from where samples were taken out during stability studies. The third column indicates the assay of the samples. The fourth column indicates dofetilide known impurity A. The fifth column indicates single unknown maximum impurity. The sixth column indicates total impurities observed in the sample. The seventh column indicates osmolality of the solution. The final eight column indicates pH of the formulation observed during the stability studies

TABLE 1
Stability results summary - Formulation 1 (Clear vials)

| | | | | Related Substances (by HPLC) | | | | |
|---|---|---|---|---|---|---|---|---|
| | | | | | % | | | |
| | | | % | % | Impurity | | | |
| | | | Assay | dofetilide | Single | % Total | | |
| Time- | | Stability | (by | RCA | MAX | impurities | | |
| points | | Condition | HPLC) | (w/w) | (w/w) | (w/w) | Osmolality | pH |
| Initial | | Initial | 99.4 | N/D | 0.14 | 0.14 | 297 | 5.57 |
| | | (T0) | | (<0.05) | (RRT | | | |
| | | | | | 1.060) | | | |
| 2 | W | 40° C./ | 99.2 | 0.07 | 0.16 | 0.28 | 297 | 5.56 |
| | | 75% RH | | | (RRT | | | |
| | | | | | 1.060) | | | |
| 4 | W | | 99.9 | 0.08 | 0.12 | 0.20 | 296 | 5.58 |
| | | | | | (RRT | | | |
| | | | | | 1.060) | | | |
| 12 | W | | 98.3 | 0.22 | 0.13 | 0.35 | 298 | 5.57 |
| | | | | | (RRT | | | |
| | | | | | 1.060) | | | |
| 24 | W | | 98.2 | 0.37 | 0.12 | 0.49 | 296 | 5.73 |
| | | | | | (RRT | | | |
| | | | | | 1.060) | | | |

TABLE 2
Stability results summary - Formulation 1 (Amber vials)

| | | | | Related Substances (by HPLC) | | | | |
|---|---|---|---|---|---|---|---|---|
| | | | | | % | | | |
| | | | % | % | Impurity | | | |
| | | | Assay | dofetilide | Single | % Total | | |
| Time- | | Stability | (by | RCA | MAX | impurities | | |
| points | | Condition | HPLC) | (w/w) | (w/w) | (w/w) | Osmolality | pH |
| Initial | | Initial | 99.4 | N/D | 0.14 | 0.14 | 297 | 5.57 |
| | | (T0) | | (<0.05) | (RRT | | | |
| | | | | | 1.060) | | | |
| 2 | W | 40° C./ | 99.6 | 0.07 | 0.12 | 0.24 | 296 | 5.55 |
| | | 75% RH | | | (RRT | | | |
| | | | | | 1.060) | | | |
| 4 | W | | 99.9 | 0.09 | 0.09 | 0.18 | 296 | 5.55 |
| | | | | | (RRT | | | |
| | | | | | 1.060) | | | |
| 12 | W | | 98.2 | 0.17 | 0.14 | 0.31 | 297 | 5.53 |
| | | | | | (RRT | | | |
| | | | | | 1.060) | | | |
| 24 | W | | 98.6 | 0.27 | 0.13 | 0.40 | 297 | 5.69 |
| | | | | | (RRT | | | |
| | | | | | 1.060) | | | |

N/D—Not detected (below quantifiable limit: 0.05%)
RCA—dofetilide Related Compound A (USP)

### Formulation Example 2

TABLE 3
Stability results summary - Formulation 2 (Clear vials)

| | | | | Related Substances (by HPLC) | | | | |
|---|---|---|---|---|---|---|---|---|
| | | | | | % | | | |
| | | | % | % | Impurity | % | | |
| | | | Assay | dofetilide | Single | Total | | |
| Time- | | Stability | (by | RCA | MAX | impurities | | |
| points | | Condition | HPLC) | (w/w) | (w/w) | (w/w) | Osmolality | pH |
| Initial | | Initial | 100.2 | N/D | 0.13 | 0.13 | 297 | 5.57 |
| | | (T0) | | (<0.05) | (RRT | | | |
| | | | | | 1.060) | | | |
| 2 | W | 40° C./ | 100.3 | 0.05 | 0.16 | 0.21 | 297 | 5.54 |
| | | 75% RH | | | (RRT | | | |
| | | | | | 1.060) | | | |
| 4 | W | | 100.6 | 0.05 | 0.11 | 0.16 | 297 | 5.57 |
| | | | | | (RRT | | | |
| | | | | | 1.060) | | | |
| 12 | W | | 98.8 | 0.15 | 0.11 | 0.26 | 298 | 5.56 |
| | | | | | (RRT | | | |
| | | | | | 1.060) | | | |
| 24 | W | | 99.4 | 0.24 | 0.13 | 0.37 | 297 | 5.67 |
| | | | | | (RRT | | | |
| | | | | | 1.060) | | | |

TABLE 4
Stability results summary - Formulation 2 (Amber vials)

| | | | | Related Substances | | | | |
|---|---|---|---|---|---|---|---|---|
| | | | | (by HPLC) | | | | |
| | | | | | % | | | |
| | | | % | % | Impurity | % | | |
| | | | Assay | dofetilide | Single | Total | | |
| Time- | | Stability | (by | RCA | MAX | impurities | | |
| points | | Cond-ition | HPLC) | (w/w) | (w/w) | (w/w) | Osmolality | pH |
| Initial | | Initial | 100.2 | N/D | 0.13 | 0.13 | 297 | 5.57 |
| | | (T0) | | (<0.05) | (RRT | | | |
| | | | | | 1.060) | | | |
| 2 | W | 40° C./ | 100.2 | 0.06 | 0.14 | 0.25 | 297 | 5.54 |
| | | 75% RH | | | (RRT | | | |
| | | | | | 1.060) | | | |
| 4 | W | | 100.7 | 0.07 | 0.12 | 0.19 | 297 | 5.55 |
| | | | | | (RRT | | | |
| | | | | | 1.060) | | | |
| 12 | W | | 99.0 | 0.13 | 0.11 | 0.24 | 297 | 5.55 |
| | | | | | (RRT | | | |
| | | | | | 1.060) | | | |
| 24 | W | | 99.5 | 0.19 | 0.13 | 0.32 | 296 | 5.68 |
| | | | | | (RRT | | | |
| | | | | | 1.060) | | | |

N/D—Not detected (below quantifiable limit: 0.05%)
RCA—dofetilide Related Compound A (USP)

Numerous modifications and variations of the present invention are possible in light of the above teachings. It is therefore to be understood that within the scope of the appended claims, the invention may be practiced otherwise that as specifically described herein.

1. A dofetilide pharmaceutical composition, comprising:
a) 10-1000 g/mL dofetilide;
b) 4-8 mg/mL sodium chloride;
c) 0.1-0.5 mg/mL acetic acid;
d) 2-6 mg/mL sodium acetate; and
e) water;
wherein:
the pH of the composition is from 4-7; and,
the composition is suitable for intravenous administration.

2. The composition of claim 1, wherein the composition, comprises:
a) 10-500 μg/mL dofetilide;
b) 5-8 mg/mL sodium chloride;
c) 0.2-0.4 mg/mL acetic acid;
d) 3-5 mg/mL sodium acetate; and
e) water;
wherein:
the pH of the composition is from 5-6; and,
the composition is suitable for intravenous administration.

3. The composition of claim 1, wherein the composition, comprises:
a) 10-100 μg/mL dofetilide;
b) 6-7 mg/mL sodium chloride;
c) 0.25-0.35 mg/mL acetic acid;
d) 3.5-4.5 mg/mL sodium acetate; and
e) water;
wherein:
the pH of the composition is from 5.2-5.8; and,
the composition is suitable for intravenous administration.

4. The composition of claim 1, wherein the composition, comprises:
a) 40-60 μg/mL dofetilide;
b) 6.3-6.7 mg/mL sodium chloride;
c) 0.3-0.35 mg/mL acetic acid;
d) 3.5-4 mg/mL sodium acetate; and
e) water;
wherein:
the pH of the composition is from 5.4-5.6; and,
the composition is suitable for intravenous administration.

5. The composition of claim 1, wherein the composition, comprises:
a) 50 μg/mL dofetilide;
b) 6.5 mg/mL sodium chloride;
c) 0.31 mg/mL acetic acid;
d) 3.7 mg/mL sodium acetate; and
e) water;
wherein:
the pH of the composition is from 5.4-5.6; and,
the composition is suitable for intravenous administration.

6. The composition of claim 1, wherein the concentration of dofetilide is 50 μg/mL.

7. The composition of claim 1, wherein the composition has an osmolality of 285-320 mOsmol/L as determined by point depression according to USP 788.

8. The composition of claim 1, wherein the composition is a solution.

9. The composition of claim 1, wherein the composition has a storage stability of at least 6 months as measured at 40° C. and 75% Relative Humidity.

10. The composition of claim 5, wherein the composition consists essentially of:
a) 50 μg/mL dofetilide;
b) 6.5 mg/mL sodium chloride;
c) 0.31 mg/mL acetic acid;
d) 3.7 mg/mL sodium acetate; and,
e) water;
wherein:
less than 1.00% w/w dofetilide related compound A (USP) is present;
the pH of the composition is from 5.4-5.6 and is optionally obtained by the addition of NaOH or HCl; and,
the composition is suitable for intravenous administration.

11. The composition of claim 10, wherein the composition has an osmolality of 285-320 mOsmol/L as determined by point depression according to USP 788.

12. The composition of claim 10, wherein the composition is a solution.

13. The composition of claim 10, wherein the composition has a storage stability of at least 6 months as measured at 40° C. and 75% Relative Humidity.

14. The composition of claim 5, wherein the composition consists of:
a) 50 μg/mL dofetilide;
b) 6.5 mg/mL sodium chloride;
c) 0.31 mg/mL acetic acid;
d) 3.7 mg/mL sodium acetate; and,
e) water;
wherein:
less than 1.00% w/w dofetilide related compound A (USP) is present;
the pH of the composition is from 5.4-5.6 and is optionally obtained by the addition of NaOH or HCl; and,
the composition is suitable for intravenous administration.

15. The composition of claim 14, wherein the composition has an osmolality of 285-320 mOsmol/L as determined by point depression according to USP 788.

16. The composition of claim 14, wherein the composition is a solution.

17. The composition of claim 14, wherein the composition has a storage stability of at least 6 months as measured at 40° C. and 75% Relative Humidity.

18. A method of manufacturing a composition of claim 1, comprising the steps of providing dofetilide with a particle size D(90) less than 10 microns.

19. A container comprising a composition of claim 1, wherein the container is amber colored glass.

20. A method of treatment of a patient in need of dofetilide therapy, comprising the step of:
a) diluting a composition of claim 1 in normal saline 0.09% or dextrose 5%; and,
b) intravenous administering the diluted composition to the patient.

21. The composition of claim 1, wherein the composition, consists essentially of:
a) 40-60 μg/mL dofetilide;
b) 6.3-6.7 mg/mL sodium chloride;
c) 0.3-0.35 mg/mL acetic acid;
d) 3.5-4 mg/mL sodium acetate; and
e) water;
wherein:
the pH of the composition is from 5.4-5.6; and,
the composition is suitable for intravenous administration.

22. The composition of claim 1, wherein the composition, consists of:
a) 40-60 μg/mL dofetilide;
b) 6.3-6.7 mg/mL sodium chloride;
c) 0.3-0.35 mg/mL acetic acid;
d) 3.5-4 mg/mL sodium acetate; and
e) water;
wherein:
the pH of the composition is from 5.4-5.6; and,
the composition is suitable for intravenous administration.
